SEQUENCE LISTING (5 sequences)

SEQ ID NO: 1 - DNA, 26 bp, Artificial Sequence
  Oligonucleotide
gannnngcnggngayggnacnacnac

SEQ ID NO: 2 - DNA, 26 bp, Artificial Sequence
  Oligonucleotide
yknykntcnccraanccnggngcytt

SEQ ID NO: 3 - DNA, 28 bp, Artificial Sequence
  Oligonucleotide
gacaaagtcggtaaagaaggcgttatca

SEQ ID NO: 4 - DNA, 1647 bp, Escherichia coli
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 5 - DNA, 1647 bp, Salmonella enterica
atggcagctaaagacgtaaaattcggtaacgacgctcgtgtgaaaatgctgcgcggcgtaaacgtactggcagatgcagtgaaagtaaccctcggtccgaaaggccgtaacgtggttctggataaatctttcggtgcgccgactatcactaaagatggtgtttccgtagcgcgtgaaatcgagctggaagacaagtttgaaaacatgggcgcgcagatggtgaaagaagttgcctctaaagcgaacgacgctgcaggcgacggcaccaccaccgcgaccgtactggcgcagtccatcattaccgaaggcctgaaagccgttgctgcgggcatgaacccgatggacctgaaacgtggtatcgacaaagcggttgctgctgcggttgaagagctgaaggcgctgtccgtaccgtgctccgactctaaagcgattgctcaggtaggtactatttccgctaactccgacgaaaccgtaggtaaactgatcgcggaagcgatggataaagtcggtaaagaaggcgtcatcactgttgaagacggtaccggtctgcaggacgaactggacgtggttgaaggtatgcagtttgaccgcggctacctgtctccttacttcatcaacaagccggaaactggcgcagtagaactggaaagcccgttcatcctgctggctgataagaaaatctccaacatccgcgaaatgctgccggttctggaagccgttgcaaaagcaggcaaaccgctgctgatcatcgctgaagatgttgaaggcgaagcgctggctaccctggtagtgaacaccatgcgtggcatcgtgaaagtggctgcggttaaagcaccgggcttcggcgatcgtcgtaaggcgatgctgcaggatatcgctaccctgaccggcggtaccgtaatctctgaagagatcggtatggagctggaaaaagcaaccctggaagacctgggtcaggcgaaacgtgttgtgatcaacaaagacaccaccaccatcattgatggcgtgggtgaagaagctgccatccagggccgtgttgctcagatccgtcagcagattgaagaagcgacctccgactacgatcgtgaaaaactgcaggagcgcgtagcgaaactggcaggcggcgttgcggtaatcaaagttggcgctgcgaccgaagttgaaatgaaagagaagaaagcccgcgttgaagatgccctgcacgcgacccgtgctgcggtagaagaaggcgtggttgctggtggtggcgttgcgctgatccgcgttgcttctaaaattgctgacctgaaaggccagaacgaagaccagaacgtgggtatcaaagttgcgctgcgcgcaatggaagctccgctgcgtcagatcgtgctgaactgcggcgaagagccgtctgttgtcgctaacaccgttaaaggcggcgacggtaactacggttacaacgcagcaactgaagaatacggcaacatgatcgatatgggtatcttggacccaaccaaagttacccgttctgcgctgcaatacgcggcttctgtggctggtctgatgatcactaccgagtgcatggtgaccgacctgccgaaaagcgatgctcctgatttaggcgctgctggcggcatgggtggtatgggtggtatgggcggcatgatgtaa

(19) United States
(12) Patent Application Publication
(10) Pub. No.: US 2004/0259226 A1
(43) Pub. Date: Dec. 23, 2004

(54) Monitoring for and detecting microbes used in bioterrorism

(76) Inventors: W. Wade Robey, Excelsior, MN (US); Alison M. Jones, Eden Prairie, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/449,460

(22) Filed: May 30, 2003

(51) Int. Cl. C12Q 1/68; C12N 1/20

(52) U.S. Cl. 435/252.3

(57) ABSTRACT

The invention provides for methods of determining the presence, absence, or amount of a microbe used in bioterrorism by detecting the presence or absence of a cpn60 marker in a sample.

### CROSS REFERENCE TO RELATED APPLICATIONS

This application claims the benefit of priority of U.S. application Ser. No. 10/392,041, filed Mar. 18, 2003, and U.S. application Ser. No. 10/306,113, filed Nov. 27, 2002.

### TECHNICAL FIELD

This invention relates to monitoring for and detecting microbes, specifically those microbes used in bioterrorism, by detecting microbial markers.

### BACKGROUND

Bioterrorism can be defined as the use, or threatened use, of biological agents to promote or spread fear or intimidation upon an individual, a specific group, or the population as a whole for religious, political, ideological, financial, or personal purposes. The biological agents generally used in bioterrorism are typically found in nature, but can be weaponized to enhance their virulence in humans and make them resistant to vaccines and antibiotics. Weaponizing can involve using selective pressure or genetic engineering to mutate or modify the genetic composition of the agent. Bioterrorism agents may be disseminated by various methods, including aerosolization, through specific blood-feeding insects, or by contaminating food or water supplies. Representative microbes that are used as biological agents in bioterrorism include the following.

Anthrax

Bacillus anthracis, the organism that causes anthrax, derives its name from the Greek word for coal, anthracis, because of its ability to cause black, coal-like cutaneous eschars. In the second half of this century, anthrax was developed as part of a larger biological weapons program by several countries, including the Soviet Union and the U.S. The number of nations believed to have biological weapons programs has steadily risen from 10 in 1989 to 17 in 1995, but how many nations are working with anthrax is uncertain. Perhaps more insidious is the specter of autonomous groups with ill intentions of using anthrax in acts of terrorism. The Aum Shinrikyo religious sect, infamous for releasing sarin gas in a Tokyo subway station in 1995, developed a number of biological weapons, including anthrax. The largest experience with inhalation anthrax occurred after the accidental release of aerosolized anthrax spores in 1979 at a military biology facility in Sverdlovsk, Russia. Some 79 cases of inhalation anthrax were reported, of which 68 were fatal.

A 1970 analysis by the World Health Organization (WHO) concluded that the release of aerosolized anthrax upwind of a population of 5,000,000 could lead to an estimated 250,000 casualties, of whom as many as 100,000 could be expected to die. The aerosol cloud would be colorless, odorless and invisible following its release. Given the small size of the spores, people indoors would receive the same amount of exposure as people on the street. There are currently no atmospheric warning systems to detect an aerosol cloud of anthrax spores. The first sign of a bioterrorism attack would most likely be patients presenting with symptoms of inhalation anthrax. One of the major problems with anthrax spores is the potentially long incubation period of subsequent infections. Exposure to aerosolized anthrax spores could cause symptoms as soon as 2 days after exposure. However, illness could also develop as late as 6-8 weeks after exposure. Further, the early presentation of anthrax disease would resemble a fever or cough, and would therefore be exceedingly difficult to diagnose without a high degree of suspicion. Once symptoms begin, death can follows 1-3 days later. If appropriate antibiotics are not started before development of symptoms, the mortality rate is estimated to be 90%.

Botulism

Botulinum toxin is derived from Clostridium botulinum. Seven antigenic types of C. botulinum toxin exist, designated A through G. They can be identified based on antibody cross reactivity studies, e.g., anti-A toxin antibodies do not neutralize the B through G toxins. Botulinum toxin poses a major threat because of its extreme potency and lethality; its ease of production, transport and misuse; and the potential need for prolonged intensive care in affected persons. Botulinum toxin is the single most poisonous substance known. A number of nations and groups have developed or are developing botulinum toxin as a biological weapon. Aum Shinrikyo tried but failed to use botulinum toxin as a biological weapon. Naturally occurring botulism results from the absorption of botulinum toxin into the circulation through the mucosal surface (e.g., the gut, or a lung) or a wound. Botulinum toxin does not penetrate intact skin. The toxin irreversibly binds to peripheral cholinergic synapses, preventing the release of the neurotransmitter acetylcholine from the terminal end of motor neurons. This leads to muscle paralysis, and in severe cases, can lead to a need for mechanical respiration.

The incubation period for food-borne botulism can be from 2 hours to 8 days after ingestion, depending on the dose of the bacteria or the toxin. The average incubation period is 12-72 hours after ingestion. Patients with botulism typically present with difficulty speaking, seeing and/or swallowing. Prominent neurologic findings in all forms of botulism include ptsosis, diplopia, blurred vision, dysarthria and dysphagia. Patients typically are afebrile and do not have an altered level of consciousness. Patients may initially present with gastrointestinal distress, nausea, and vomiting that precede the neurological symptoms. Symptoms are similar for all toxin types, but the severity of illness can vary widely, in part depending on the amount of toxin absorbed. Recovery from paralysis can take from weeks to months and requires the growth of new motor nerve endings. Natural cases of botulism are rare and typically result from food contamination. Many types of food have been associated with outbreaks in the past. The largest botulism outbreak in the U.S. in the past century occurred in 1977, when 59 people became ill from poorly preserved jalapeño peppers.

Plague

Plague, the disease caused by the bacteria Yersinia pestis, has had a profound impact on human history. In A.D. 541, the first great plague pandemic began in Egypt and swept over the world in the next four years. Population losses attributable to the plague during those years were between 50% and 60%. In 1346, the second plague pandemic, also known as the Black Death or the Great Pestilence, erupted and within 5 years, had ravaged the Middle East and killed more than 13 million in China and 20-30 million (one third of the population) in Europe. Plague is one of very few diseases that can create widespread panic following the discovery of even a small number of cases. This was apparent in Surat, India in 1994, when an estimated 500,000 persons fled the city in fear of a plague epidemic.

In the 1950s and 1960s, the U.S. and Soviet biological weapons programs developed techniques to directly aerosolize plague particles, a technique that leads to pneumonic plague, an otherwise uncommon, highly lethal and potentially contagious form of plague. A modern bioterrorism attack would most probably occur via aerosol dissemination of Y. pestis, and the ensuing outbreak would be almost entirely pneumonic plague. A 1970 WHO assessment asserted that, in a worst-case scenario, a dissemination of 50 kg of Y. pestis in an aerosol cloud over a city of 5 million might result in 150,000 cases of pneumonic plague, 80,000-100,000 of which would require hospitalization, and 36,000 of which would be expected to die.

Tularemia

Francisella tularensis, the organism that causes tularemia, is one of the most infectious pathogenic bacteria known, requiring inoculation or inhalation of as few as 10 organisms to cause disease. It is considered to be a dangerous potential biological weapon because of its extreme infectivity, ease of dissemination, and substantial capacity to cause illness and death. During World War II, the potential of F. tularensis as a biological weapon was studied by the Japanese as well as by the U.S. and its allies. Tularemia was one of several biological weapons that were stockpiled by the U.S. military in the late 1960's, all of which were destroyed by 1973. The Soviet Union continued weapons production of antibiotic and vaccine-resistant strains into the early 1990s.

F. tularensis is a hardy, non-spore forming organism that is capable of surviving for weeks at low temperatures in water, moist soil, hay, straw or decaying animal carcasses. F. tularensis has been divided into two subspecies: F. tularensis biovar tularensis (type A), which is the most common biovar isolated in North America and may be highly virulent in humans and animals; and F. tularensis biovar palaearctica (type B) which is relatively avirulent and thought to the cause of all human tularemia in Europe and Asia. A WHO expert committee reported in 1970 that if 50 kg of virulent F. tularensis was dispersed as an aerosol over a metropolitan area with a population of 5 million, there would an estimated 250,000 incapacitating casualties, including 19,000 deaths. Aerosol dissemination of F. tularensis in a populated area would be expected to result in the abrupt onset of large numbers of cases of acute, non-specific febrile illness beginning 3 to 5 days later (incubation range, 1-14 days), with pleuropneumonitis developing in a significant proportion of cases over the ensuing days and weeks. Without antibiotic treatment, the clinical course could progress to respiratory failure, shock and death.

In addition to those microbes discussed above, other microbes that may not have a lethal effect can be used as biological agents in bioterrorism. Such microbes include E. coli, or Salmonella spp., which could be used to infect a large population of individuals and make them sick. For example, in 1984 in The Dalles, Oregon, followers of Bhagwan Shree Rajneesh contaminated numerous salad bars in restaurants with Salmonella, resulting in 751 people becoming ill.

Further, there are numerous microbes that can be used in agricultural bioterrorism such as Liberobacter africanus, Liberobacter asiaticus, Peronosclerospora philippinesis, Phakospsora pachyrhizi, Ralstonia solanacearum, race 3, biovar 2, Sclerophthora rayssiae var. zeae, Synchytrium endobioticum, Xanthomonas oryzae pv. oryzicola, and Xylella fastidiosa (citrus variegated chlorosis strain). See, for example, the Agriculture Bioterrorism Protection Act of 2002 (7 C.F.R. §331). Therefore, a number of microbes can be used in bioterrorism.

### SUMMARY

The invention is based on the discovery that the presence or absence of a microbe used in bioterrorism can be determined quickly and sensitively by detecting the presence and/or concentration of a microbial marker, specifically a cpn60 marker, in a sample. Chaperonin 60 (cpn60) markers are particularly useful for determining the presence of a microbe in a sample and for optionally identifying the microbe. Chaperonin proteins are molecular chaperones required for proper folding of polypeptides in vivo. cpn60 is found universally in prokaryotes and in the organelles of eukaryotes, and can be used as a species-specific target and/or probe for identification and classification of microbes. Sequence diversity within cpn60 appears greater between and within bacterial genera than for 16S rDNA sequences, thus making cpn60 a superior target sequence having more distinguishing power for microbial identification at the species level than 16S rDNA sequences.

In one aspect, the invention provides a method for detecting the presence, absence, or amount of one or more microbes used in bioterrorism. Such a method includes a) providing a sample obtained from an environment susceptible to bioterrorism attack or an environment within which a bioterrorism attack has taken place; and b) detecting the presence, absence, or amount of a cpn60 marker in the sample, wherein the cpn60 marker is specific for one or more of the microbes used in bioterrorism. Generally, the presence of the cpn60 marker in the sample is indicative of the presence, in the environment, of one or more of the microbes used in bioterrorism.

The detecting step can be capable of identifying the microbe(s) in the sample, and further can be capable of quantitating the microbe(s) in the sample. The detecting step can be a nucleic acid-based assay such as PCR or FISH assays. The detecting step also or alternatively can be a polypeptide-based assay such as an immunodiagnostic assay (e.g., ELISA), a mass spectrometric technique, or a surface plasmon resonance technique.

In some embodiments, the sample is provided at two or more points, which can be time points or location points. In these embodiments, the method of the invention can further include comparing the presence, absence, or amount of the microbe at the two or more points.

In some embodiments, the method of the invention can further include acquiring a control sample from one or both of the environments. In addition, the method of the invention can still further include comparing the presence, absence, or amount of microbes used in bioterrorism in the control sample with the presence, absence, or amount of the microbes used in bioterrorism in the sample.

Typically, the cpn60 marker is a cpn60 nucleic acid or a cpn60 polypeptide. Embodiments in which the cpn60 marker is a cpn60 nucleic acid, the cpn60 nucleic acid can be a nucleic acid coding sequence of a cpn60 protein. Typically, the cpn60 nucleic acid is an amplified sequence of a cpn60 coding sequence from the microbes used in bioterrorism.

Representative microbes used in bioterrorism and that can be detected by the method of the invention include B. anthracis, C. botulinum, Y. pestis, and F. tularensis.

Representative environments susceptible to bioterrorism attack can include a food-service facility, a water facility, a transportation facility, an entertainment facility, a shopping mall, and an office building. Examples of a food-service facility include a restaurant, a cafeteria, a snack bar, and a convenience store. Examples of a water facility include potable water facilities, desalinization facilities, dams, recycled water facilities, water storage tanks, and potable water reservoirs. Examples of a transportation facility include an airport, a bus terminal, a train terminal, a port, a Custom's checkpoint, and an immigration checkpoint. Examples of an entertainment facility include a club, a theater, a stadium, and an arena.

Typically, a sample can be a biological sample or a non-biological sample. Biological samples include water, air, food, a tissue sample from an animal or human, or a fluid sample from an animal or human. Non-biological samples can be collected, for example, from a fomite.

In another aspect, the invention provides an article of manufacture that includes at least one cpn60 antibody and an indicator molecule. Typically, the cpn60 antibody has specific binding affinity for a cpn60 polypeptide from a microbe used in bioterrorism. Generally, the cpn60 antibody is attached to a solid support such as a dipstick. An article of manufacture of the invention can further include instructions for using the cpn60 antibody to detect a cpn60-containing microbe used in bioterrorism.

In another aspect, the invention provides an article of manufacture that includes at least one cpn60 oligonucleotide, and instructions therein for using the cpn60 oligonucleotide(s) to determine, in a sample, the presence, absence, or amount of a microbe used in bioterrorism. Generally, the cpn60 oligonucleotide is complementary to cpn60 nucleic acids from at least one microbe used in bioterrorism. One or more cpn60 oligonucleotide(s) can be attached to a microarray in an article of manufacture of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is the sequence of a cpn60 gene from E. coli (SEQ ID NO:4; GenBank Accession No. NC_{—}000913). Sequences to which the universal cpn60 primers described herein can hybridize (or the complement thereof) are underlined.

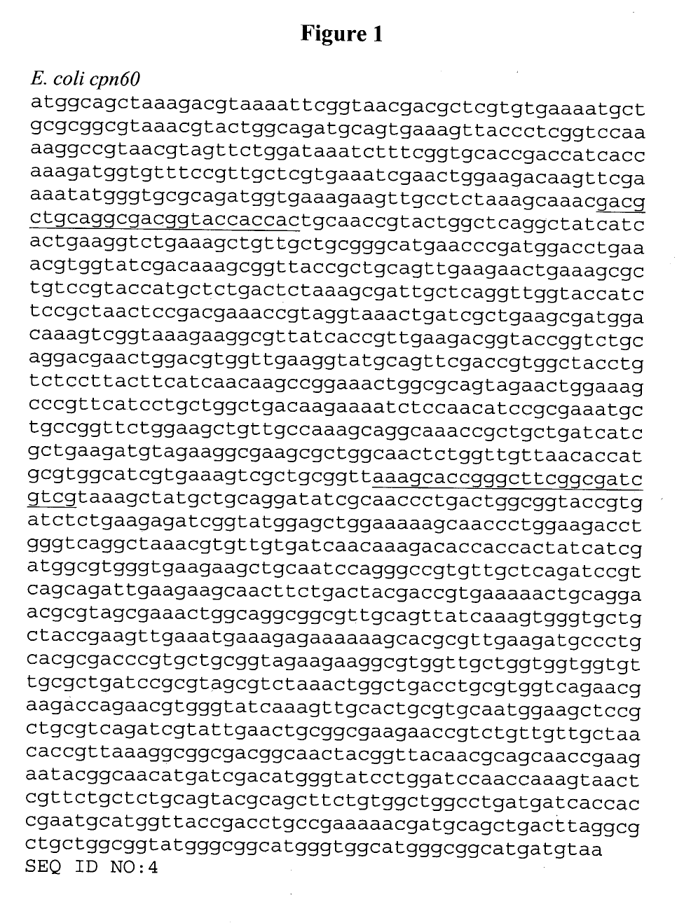

FIG. 2 is the sequence of a cpn60 gene from Salmonella enterica (SEQ ID NO:5; GenBank Accession No. NC_{—}003198). Sequences to which the universal cpn60 primers described herein can hybridize (or the complement thereof) are underlined.

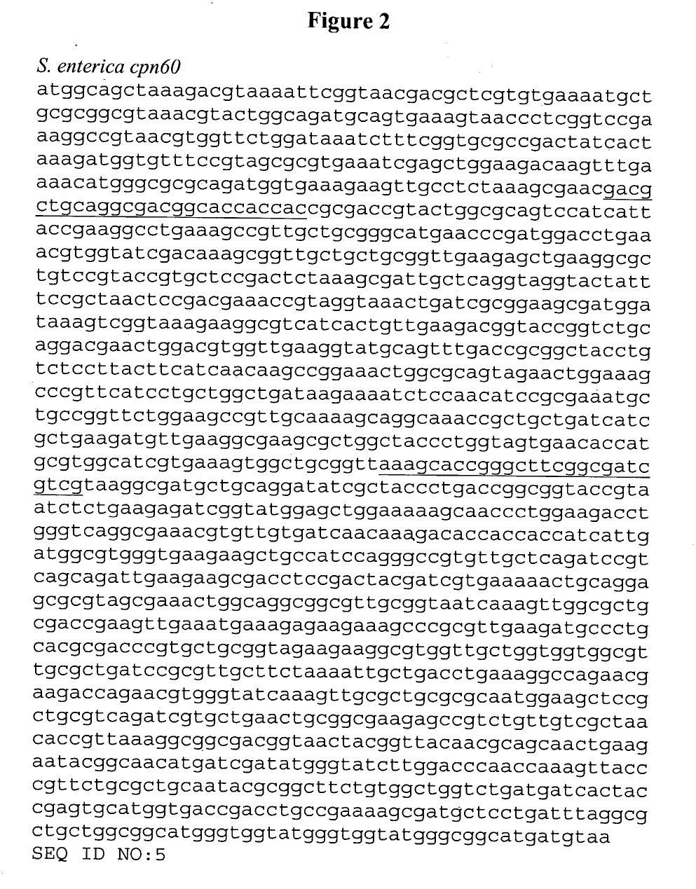

Like reference symbols in the various drawings indicate like elements.

### DETAILED DESCRIPTION

The present invention provides methods of monitoring for and detecting the presence or absence of one or more microbes used in bioterrorism. In particular, the presence or absence of a microbe used in bioterrorism can be determined quickly and sensitively by detecting the presence and/or concentration of a microbial marker, specifically a cpn60 marker, in a sample.

Environments Susceptible to Bioterrorism Attack

Since biological agents used in terrorism can be released using a variety of different means and methods, the environments that are susceptible to bioterrorism attack are numerous. As used herein, environments that are susceptible to bioterrorism attack include but are not limited to the following: a food-service facility, a water facility, a transportation facility, an entertainment facility, a shopping mall, and an office building. In addition, the air within such facilities or buildings provides an environment susceptible to bioterrorism attack, as does the atmospheric air outside.

Food-service facilities include such places as restaurants, cafeterias, snack bars, and convenience stores. Water facilities can include potable water facilities, desalinization facilities, dams, recycled water facilities, water storage tanks, and potable water reservoirs (e.g., water coolers). Non-limiting examples of transportation facilities include airports, bus terminals, train terminals, ports, Custom's checkpoints, and immigration checkpoints. According to the invention, a transportation facility also can include the means for transportation used or housed within such a facility, such as a car, a bus, a plane, a train, or a ship. Entertainment facilities can include, without limitation, nightclubs, theaters, stadiums, and arenas. With respect to shopping malls and office buildings, it is likely that the larger facilities (e.g., megamalls, or high rise office buildings) with more incoming and outgoing traffic would present a more desirable target for a bioterrorism attack, although the invention is not limited to facilities having a minimum size or occupancy number.

The methods of the invention for detecting the presence, absence, or amount of microbes used in bioterrorism also can be used to evaluate and confirm false or hoax claims of bioterrorism. Methods of the invention for rapidly evaluating claims of bioterrorism that may be false can significantly reduce the panic that may ensue following a claim of bioterrorism, as well as reduce or eliminate the unnecessary mobilization of response and medical teams.

Sample Types and Sampling Methods

The methods described herein are capable of detecting the presence, absence, or amount of a microbe, and optionally identifying the microbe, based on the presence of a cpn60 marker in a sample. A sample can be collected from high-risk areas such as air, food, and transportation and entertainment facilities to monitor for a bioterrorism attack. After a bioterrorism attack has occurred, however, samples can be collected from anything in the vicinity of the attack such as surfaces, objects, and individuals. Although by no means limiting, samples generally can be classified as “biological” and “non-biological.” A biological sample can refer to any sample obtained, directly or indirectly, from an animal or a human. A biological sample also can refer to environmental samples such as those obtained from food (prepared or raw), water, or air.

Representative biological samples that can be obtained from an animal or a human include or are derived from biological tissues (e.g., skin and hair), biological fluids (e.g., blood, urine, and saliva), and biological elimination products (e.g., feces). Biological tissues can include biopsy samples from, for example, the gastrointestinal tract, the mucous membrane, the mouth, the nose, and the skin, or swabs of the biological tissue of interest, e.g., nasal swabs, throat swabs, dermal swabs. The tissue can be any appropriate tissue from a human, but also can be derived from an animal, such as a cow, a pig, a horse, a goat, a sheep, a dog, a cat, or a bird.

Biological fluids can include bodily fluids (e.g., urine, milk, lachrymal fluid, vitreous fluid, sputum, cerebrospinal fluid, sweat, lymph, saliva, semen, blood, or serum or plasma derived from blood); a lavage such as a lung lavage, or a gastric lavage; an aspirate; a fluid such as a cell culture or a supernatant from a cell culture; and a fluid such as a buffer that has been used to obtain or resuspend a sample, e.g., to wash or to wet a swab in a swab sampling procedure. Biological samples can be obtained from a human or an animal using methods and techniques known in the art. See, for example, Diagnostic Molecular Microbiology: Principles and Applications (Persing et al., eds, 1993, American Society for Microbiology, Washington D.C.).

In addition, methods are known for obtaining samples from the environment. An air or water sample can be collected using known methods and equipment. Air and/or water samples can be collected periodically over time (e.g., hours, days, weeks, or months) or over multiple regions (e.g., multiple locations within a city, or multiple locations around a country) to monitor for microbes used in bioterrorism. A biological sample can also be a food sample. For example, the sample may be a prepared food sample, e.g., from a restaurant. Such a prepared food sample may be either cooked or raw (e.g., salads, juices, or fruits). In other embodiments, the food sample may be unprocessed and/or raw. The food sample may be perishable. Typically, food samples will be taken from food products such as beef, pork, poultry, seafood, dairy, fruit, vegetable, seed, nut, fungus, and grain. Dairy food samples include milk, eggs, butter, and cheese, as well as sauces and condiments made from the same.

The methods of the present invention also can be used to detect the presence of microbes in or on “non-biological” samples. As used herein, a “non-biological” sample includes any sample taken from, for example, an inanimate object or a solid surface. For example, a fomite may be sampled to detect the presence or absence of a microbe. A fomite is a physical inanimate object onto which a microbe can settle or be deposited. Representative fomites from, for example, an entertainment or a transportation facility include the surface of floors, seats, or serving areas. Additional non-limiting examples of fomites include utensils, drinking glasses, food processing equipment, cutting surfaces, floors, ceilings, walls, drains, ventilation systems, machines, door handles, and clothes.

A microbe may be left as a residue on a fomite. In such cases, it is important to accurately detect the presence of the microbe on the fomite. For example, it is known that microbes may exist in viable but non-culturable forms on fomites, or that non-culturable bacteria of selected species can be resuscitated to a culturable state under certain conditions. Often such non-culturable bacteria are present in biofilms on fomites. Accordingly, detection methods that rely on culturable forms may significantly under-report microbial contamination on fomites. The methods of the present invention, specifically PCR-based methods, can aid in the detection of microbes, particularly non-culturable forms, by amplification and detection of cpn60 nucleic acid sequences.

Methods for collecting and storing biological and non-biological samples are generally known to those of skill in the art. For example, the Association of Analytical Communities International (AOAC International) publishes and validates sampling techniques for testing foods and agricultural products for microbial contamination. See also WO 98/32020 and U.S. Pat. No. 5,624,810, which set forth methods and devices for collecting and concentrating microbes from the air, liquid, or a surface. WO 98/32020 also provides methods for removing somatic cells or animal body cells present at varying levels in certain samples.

In particular embodiments of the methods described herein, a separation and/or concentration step may be necessary to separate any microbes present from other components of a sample or to concentrate the microbe to an amount sufficient for rapid detection. For example, a sample suspected of containing a biological microbe may require a selective enrichment of the microbe (e.g., by culturing in appropriate media, e.g., for 4-96 hours, or longer) prior to employing the detection methods described herein. Alternatively, appropriate filters and/or immunomagnetic separations can concentrate a microbe without the need for an extended growth stage. For example, antibodies specific for a cpn60 polypeptide can be attached to magnetic beads and/or particles. Multiplexed separations in which two or more concentration processes (e.g., centrifugation, membrane filtration, electrophoresis, ion exchange, affinity chromatography, and immunomagnetic separations) are employed also are contemplated.

Certain air or water samples may need to be concentrated. For example, certain air sampling methods require the passage of a prescribed volume of air over a filter to trap any microbes, followed by transfer into a buffer or liquid culture. Alternatively, the focused air is passed over a plate (e.g., agar) medium for culturing.

Methods for sampling a tissue or a fomite with a swab are known to those of skill in the art. Generally, a swab is hydrated (e.g., with an appropriate buffer, such as Cary-Blair medium, Stuart's medium, Amie's medium, PBS, buffered glycerol saline, or water) and used to sample an appropriate surface (a fomite or tissue) for a microbe. Any microbe present is then recovered from the swab, such as by centrifugation of the hydrating fluid away from the swab, removal of supernatant, and resuspension of the centrifugate in an appropriate buffer, or by washing of the swab with additional diluent or buffer. The so-recovered sample may then be analyzed according to the methods described herein for the presence of a microbe used in bioterrorism. Alternatively, the swab may be used to inoculate a liquid or plate (e.g., agar) medium in order to culture of any microbes present. Suitable swabs include both cotton and sponge swabs; see, for example, those provided by Tecra®, such as the Tecra ENVIROSWAB®.

The samples can be used “as is,” or may need to be treated prior to application of the detection methods employed herein. For example, samples can be processed (e.g., by nucleic acid or protein extraction methods and/or kits known in the art) to release nucleic acid or proteins. In other cases, a biological sample can be contacted directly with the appropriate reagents (e.g., PCR reaction components and appropriate oligonucleotide primers and/or probes).

Detection of cpn60 Markers

Methods provided herein are useful for determining the presence of one or more microbes used in bioterrorism and optionally for identifying the particular microbe. As used herein, a microbe used in bioterrorism includes B. anthracis, C. botulinum, Y. pestis, and F. tularensis.

The detection of a microbe in a sample (e.g., a biological sample or a non-biological sample) obtained from an environment susceptible to a bioterrorism attack or an environment in which a bioterrorism attack has occurred can be determined using methods that involve detection of a cpn60 marker. cpn60 markers include cpn60 nucleic acids and cpn60 polypeptides. As used herein, a cpn60 nucleic acid is a nucleic acid that includes, is complementary to, or specifically hybridizes to all or a portion of a cpn60 nucleic acid sequence. The term “nucleic acid” as used herein encompasses both RNA and DNA, including genomic DNA. Nucleic acid can be double-stranded or single-stranded, and can contain one or more restriction enzyme sites.

Generally, a cpn60 nucleic acid marker will be all or a portion of the nucleic acid coding sequence of a cpn60 protein. A cpn60 nucleic acid may be specific to a particular species of microbe or may be universal to multiple species of microbe. Species-specific cpn60 nucleic acid sequences are cpn60 nucleic acid sequences that hybridize preferentially to cpn60 nucleic acid sequences from a given microbial species under appropriate assay conditions. One of skill in the art can design oligonucleotides to detect such species-specific cpn60 nucleic acid sequences by e.g., aligning cpn60 nucleic acid coding sequences from multiple species and looking for regions within cpn60 sequences that vary between the species, e.g., sequences from one species that would not cross-hybridize to cpn60 nucleic acid sequences from other species under the same assay conditions. Alternatively, one of skill in the art will recognize that variable regions, e.g., those that demonstrate no more than 99% sequence similarity (e.g., no more than 85%, 86%, 87%, 88%, 89%, 90%, 91%, 92%, 93%, 94%, 95%, 96%, 97%, and 98% sequence similarity) to a cpn60 nucleic acid from another species may also be useful as species-specific cpn60 nucleic acids. Use of such species-specific oligonucleotides in the methods described herein allows for the discriminatory detection of particular microbes in a sample.

In addition, methods of the invention that use species-specific cpn60 nucleic acids can be used for epidemiology studies of, for example, the origin or source of a microbe used in a bioterrorism attack. For example, cpn60 primers or probes can be designed that discriminate between particular strains or sub-types within a species of microbe used in bioterrorism based on the presence of mutations or polymorphisms within the cpn60 sequence. Thus, methods of the invention can be used to compare microbes used in a bioterrorism attack with known sources of such microbes (depository's, stocks from countries or militant/terrorist groups known to maintain stocks of such microbes used in bioterrorism) to track down the source of a bioterrorism attack.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at http://www.ncbi.nlm.nih.gov. BLAST searches can be performed to determine percent sequence identity between a cpn60 nucleic acid sequence from one species of microbe and a cpn60 nucleic acid sequence from another species of microbe aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between cpn60 sequences, the default parameters of the respective programs are used.

Sequences of cpn60 nucleic acids from microbes used in bioterrorism can be obtained using conventional methods. For example, cpn60 sequences can be found in public databases such as GenBank. Representative nucleic acid sequences from the genome of microbes using in bioterrorism are shown in GenBank Accession Nos. NC_{—}003366, AP003193, NC_{—}003143, AJ414142, and NC_{—}003995. A representative cpn60 nucleic acid sequence from B. anthracis is shown in GenBank Accession No. AB028452. Alternatively, cpn60 nucleic acid sequences from microbes used in bioterrorism can be identified using homologous cpn60 nucleic acid sequences from other microbes in, for example, BLAST searches of public databases, or hybridization to libraries containing nucleic acids from microbes used in bioterrorism.

As used herein, a “universal” cpn60 nucleic acid is a cpn60 nucleic acid sequence that is capable of hybridizing or annealing under the appropriate assay conditions to cpn60 nucleic acid sequences from more than one species of microbe. Such sequences, of course, would not hybridize to non-cpn60 nucleic acids under the same assay conditions. One of skill in the art will recognize that hybridization assay conditions can be manipulated in a variety of ways to increase or decrease stringency, e.g., by salt, temperature, choice of buffer, etc. See e.g., Sambrook et al., Molecular Cloning; A Laboratory Manual, 2^{nd} Ed., Cold Spring Harbor Laboratory Press, 1989. Alternatively, one of skill in the art will recognize that a cpn60 nucleic acid sequence demonstrating greater than 75%, 80%, 85%, 90%, or 95% sequence identity to at least a second cpn60 nucleic acid sequence may be useful as universal cpn60 nucleic acids. For example, cpn60 coding sequences from, for example, B. anthracis, or sequences derived therefrom, may cross-hybridize under the appropriate assay conditions or have sufficiently similar sequences to cpn60 coding sequences from, for example, B. anthracis and C. botulinum. In addition, varying hybridization stringencies can be tested to ascertain optimal conditions for cross-hybridization. Designing such universal cpn60 nucleic acids allows for the use of a single set of reagents to detect the presence of more than one species of microbes.

The universality of a universal cpn60 nucleic acid can be manipulated depending on the methods used and the desired results of the assay. If, for example, the above-described universal cpn60 nucleic acids are able to detect microbes in a sample other than those used in bioterrorism, those microbes that are specifically used in bioterrorism can be specifically detected or identified using species-specific cpn60 nucleic acids. Alternatively, universal cpn60 nucleic acids can be designed to be universal with respect to only those microbes that are potentially or traditionally used in bioterrorism.

As used herein, a cpn60 polypeptide marker is a polypeptide that includes all or a portion of a cpn60 protein. As with cpn60 nucleic acids, a cpn60 polypeptide marker can be specific to a particular species of microbe or universal to more than one species. A species-specific cpn60 polypeptide marker is all or a portion of the cpn60 protein from a particular species of microbe, wherein detection of that species' cpn60 protein is specific over detection of cpn60 proteins from other species of microbes. In the methods of the present invention, the probe or analytical method for detecting the marker should be capable of discriminating between the particular cpn60 polypeptide and other cpn60 polypeptides, e.g., by mass in mass-spectrometry applications or by a particular epitope in an antibody assay. For example, and as described more fully below, one of skill in the art will recognize that antibodies, particularly monoclonal antibodies, can be obtained that recognize an epitope that is specific to a particular species' cpn60 protein. Accordingly, use of such specific antibodies in the methods described herein allows the differential detection of a particular species of microbe in a sample.

In other embodiments, a cpn60 polypeptide marker can be universal. For example, a “universal” cpn60 polypeptide marker may be a common structural (conformational) epitope present in two or more cpn60 proteins. As described more fully below, antibodies, particularly polyclonal antibodies, raised against cpn60 proteins or polypeptides can be screened for cross-reactivity to common epitopes on cpn60 polypeptides from two or more species of microbes.

Nucleic Acid-Based Assays

Nucleic acid-based methods for detecting the presence or absence of a microbe used in bioterrorism and, if desired, identifying and/or quantitating the amount of the microbe in a sample can include amplification of a cpn60 nucleic acid. Amplification methods such as PCR provide powerful means by which to increase the amount of a particular nucleic acid sequence. Nucleic acid hybridization also can be used to determine the presence or absence of a microbe in a sample. Probing amplification products with species-specific hybridization probes is one of the most powerful analytical tools available for profiling. The physical matrix for hybridization can be a nylon membrane (e.g., a macroarray) or a microarray (e.g., a microchip), incorporation of one or more hybridization probes into an amplification reaction (e.g., TaqMan® or Molecular Beacon technology), solution-based methods (e.g., ORIGEN technology), or any one of numerous approaches devised for microbial evaluation. As discussed above, probes can be designed to preferentially hybridize to amplification products from individual species or to discriminate specific species.

PCR and Real-Time PCR

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in the present invention include oligonucleotide primers capable of acting as a point of initiation of nucleic acid synthesis within or adjacent to cpn60 sequences (see below). A primer can be purified from a restriction digest by conventional methods, or can be produced synthetically. Primers typically are single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular species of microbe such as B. anthracis, or can be designed to amplify a sequence from more than one species of microbe. Primers that can be used to amplify a nucleotide sequence from more than one species are referred to herein as “universal primers.”

PCR assays can employ template nucleic acids such as DNA or RNA, including messenger RNA (mRNA). The template nucleic acid need not be purified; it can be a minor fraction of a complex mixture, such as microbial nucleic acid contained in animal cells. Template DNA or RNA can be extracted from a sample (e.g., biological or non-biological sample) using routine techniques such as those described in Diagnostic Molecular Microbiology: Principles and Applications (Persing et al., eds., 1993, American Society for Microbiology, Washington D.C.). Nucleic acids can be obtained from any of a number of sources, including plasmids, bacteria, yeast, organelles, and higher organisms such as plants and animals. Standard conditions for generating a PCR product are well known in the art (see, e.g., PCR Primer: A Laboratory Manual, Dieffenbach and Dveksler, eds., Cold Spring Harbor Laboratory Press, 1995).

Once a PCR amplification product is generated, it can be detected by, for example, hybridization using FRET technology. FRET technology (see, for example, U.S. Pat. Nos. 4,996,143, 5,565,322, 5,849,489, and 6,162,603) is based on the concept that when a donor fluorescent moiety and a corresponding acceptor fluorescent moiety are positioned within a certain distance of each other, energy transfer taking place between the two fluorescent moieties can be visualized or otherwise detected and quantitated. For example, two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product at the appropriate positions, a FRET signal is generated. Hybridization temperatures and times can range from about 35° C. to about 65° C. for about 10 seconds to about 1 minute. Detection of FRET can occur in real-time, such that the increase in an amplification product after each cycle of a PCR assay is detected and, in some embodiments, quantitated.

Fluorescent analysis and quantitation can be carried out using, for example, a photon counting epifluorescent microscope system (containing the appropriate dichroic mirror and filters for monitoring fluorescent emission in a particular range of wavelengths), a photon counting photomultiplier system, or a fluorometer. Excitation to initiate energy transfer can be carried out with an argon ion laser, a high intensity mercury arc lamp, a fiber optic light source, or another high intensity light source appropriately filtered for excitation in the desired range.

Fluorescent moieties can be, for example, a donor moiety and a corresponding acceptor moiety. As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of an acceptor fluorescent moiety typically should be at least 100 nm greater than the wavelength maximum of the excitation spectrum of the donor fluorescent moiety, such that efficient non-radiative energy transfer can be produced therebetween.

Fluorescent donor and corresponding acceptor moieties are generally chosen for (a) high efficiency Förster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen with an excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, and other chelates of Lanthanide ions (e.g., Europium, or Terbium). Donor and acceptor fluorescent moieties can be obtained from, for example, Molecular Probes, Inc. (Eugene, Oreg.) or Sigma Chemical Co. (St. Louis, Mo.).

Donor and acceptor fluorescent moieties can be attached to probe oligonucleotides via linker arms. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å in length. The linker arm may be of the kind described in WO 84/03285, for example. WO 84/03285 also discloses methods for attaching linker arms to a particular nucleotide base, as well as methods for attaching fluorescent moieties to a linker arm.

An acceptor fluorescent moiety such as an LC™-Red 640-NHS-ester can be combined with C6-Phosphoramidites (available from ABI (Foster City, Calif.) or Glen Research (Sterling, Va.)) to produce, for example, LC™-Red 640-Phosphoramidite. Linkers frequently used to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPG's that require coupling of a fluorescein-NHS-ester after oligonucleotide synthesis.

Using commercially available real-time PCR instrumentation (e.g., LightCycler™, Roche Molecular Biochemicals, Indianapolis, Ind.), PCR amplification, detection, and quantitation of an amplification product can be combined in a single closed cuvette with dramatically reduced cycling time. Since detection and quantitation occur concurrently with amplification, real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory, in the field, or at the point of care.

Conventional PCR methods in conjunction with FRET technology can be used to practice the methods of the invention. In one embodiment, a LightCycler™ instrument is used. A detailed description of the LightCycler™ System and real-time and on-line monitoring of PCR can be found at the Roche website. The following patent applications describe real-time PCR as used in the LightCycler™ technology: WO 97/46707, WO 97/46714, and WO 97/46712. The LightCycler™ instrument is a rapid thermal cycler combined with a microvolume fluorometer utilizing high quality optics. This rapid thermocycling technique uses thin glass cuvettes as reaction vessels. Heating and cooling of the reaction chamber is controlled by alternating heated and ambient air. Due to the low mass of air and the high ratio of surface area to volume of the cuvettes, very rapid temperature exchange rates can be achieved within the LightCycler™ thermal chamber. Addition of selected fluorescent dyes to the reaction components allows the PCR to be monitored in real-time and on-line. Furthermore, the cuvettes serve as an optical element for signal collection (similar to glass fiber optics), concentrating the signal at the tip of the cuvette. The effect is efficient illumination and fluorescent monitoring of microvolume samples.

The LightCycler™ carousel that houses the cuvettes can be removed from the instrument. Therefore, samples can be loaded outside of the instrument (in a PCR Clean Room, for example). In addition, this feature allows for the sample carousel to be easily cleaned and sterilized. The fluorometer, as part of the LightCycler™ apparatus, houses the light source. The emitted light is filtered and focused by an epi-illumination lens onto the top of the cuvette. Fluorescent light emitted from the sample is then focused by the same lens, passed through a dichroic mirror, filtered appropriately, and focused onto data-collecting photohybrids. The optical unit currently available in the LightCycler™ instrument (Roche Molecular Biochemicals, Catalog No. 2 011 468) includes three band-pass filters (530 nm, 640 nm, and 710 nm), providing three-color detection and several fluorescence acquisition options. Data collection options include once per cycling step monitoring, fully continuous single-sample acquisition for melting curve analysis, continuous sampling (in which sampling frequency is dependent on sample number) and/or stepwise measurement of all samples after defined temperature interval.

The LightCycler™ can be operated and the data retrieved using a PC workstation and a Windows operating system. Signals from the samples are obtained as the machine positions the capillaries sequentially over the optical unit. The software can display the presence and amount of fluorescent signals in real-time immediately after each measurement. Fluorescent acquisition time is 10-100 milliseconds (msec). After each cycling step, a quantitative display of fluorescence vs. cycle number can be continually updated for all samples. The generated data can be stored for further analysis.

Real-time PCR methods include multiple cycling steps, each step including an amplification step and a hybridization step. In addition, each cycling step typically is followed by a FRET detecting step to detect hybridization of one or more probes to an amplification product. The presence of an amplification product is indicative of the presence of one or more cpn60-containing microbes. As used herein, a “cpn60-containing microbe” refers to a microbe that contains cpn60 nucleic acid sequences. Generally, the presence of FRET indicates the presence of one or more cpn60-containing microbes in the sample, and the absence of FRET indicates the absence of cpn60-containing microbes in the sample. Typically, detection of FRET within, for example, 20, 25, 30, 35, 40, or 45 cycling steps is indicative of the presence of a cpn60-containing microbe.

As described herein, cpn60 amplification products can be detected using labeled hybridization probes that take advantage of FRET technology. A common format of FRET technology utilizes two hybridization probes that are designed to hybridize in close proximity to each other, where one probe is labeled with a donor fluorescent moiety and the other is labeled with a corresponding acceptor fluorescent moiety. Thus, two cpn60 probes can be used, one labeled with a donor fluorophore and the other labeled with a corresponding acceptor fluorophore. The presence of FRET between the donor fluorescent moiety of the first cpn60 probe and the corresponding acceptor fluorescent moiety of the second cpn60 probe is detected upon hybridization of the cpn60 probes to the cpn60 amplification product. For example, a donor fluorescent moiety such as fluorescein is excited at 470 nm by the light source of the LightCycler™ Instrument. During FRET, the fluorescein transfers its energy to an acceptor fluorescent moiety such as LightCycler™-Red 640 (LC™-Red 640) or LightCycler™-Red 705 (LC™-Red 705). The acceptor fluorescent moiety then emits light of a longer wavelength, which is detected by the optical detection system of the LightCycler™ instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules (e.g., the number of copies of cpn60).

Another FRET format can include the use of TaqMan® technology to detect the presence or absence of a cpn60 amplification product, and hence, the presence or absence of cpn60-containing microbes. TaqMan® technology utilizes one single-stranded hybridization probe labeled with two fluorescent moieties. When a first fluorescent moiety is excited with light of a suitable wavelength, the absorbed energy is transferred to a second fluorescent moiety according to the principles of FRET. In TaqMan® technology, the second fluorescent moiety generally is a quencher molecule. During the hybridization step of the PCR reaction, the labeled hybridization probe binds to the target DNA (i.e., the cpn60 amplification product) and is degraded by the 5′ to 3′ exonuclease activity of the Taq Polymerase during the subsequent elongation phase. As a result, the excited fluorescent moiety and the quencher moiety become spatially separated from one another. As a consequence of excitation of the first fluorescent moiety in the absence of the quencher, the fluorescence emission from the first fluorescent moiety can be detected. By way of example, an ABI PRISM® 7700 Sequence Detection System (Applied Biosystems, Foster City, Calif.) uses TaqMan® technology, and is suitable for performing the methods described herein for detecting cpn60-containing. Information on PCR amplification and detection using an ABI PRISM® 770 system can be found at the Applied Biosystems website (world wide web at appliedbiosystems.com/products).

Molecular beacons in conjunction with FRET also can be used to detect the presence of a cpn60 amplification product using the real-time PCR methods of the invention. Molecular beacon technology uses a hybridization probe labeled with a first fluorescent moiety and a second fluorescent moiety. The second fluorescent moiety generally is a quencher, and the fluorescent labels typically are located at each end of the probe. Molecular beacon technology uses an oligonucleotide probe having sequences that permit secondary structure formation (e.g., a hairpin). As a result of secondary structure formation within the probe, both fluorescent moieties are in spatial proximity when the probe is in solution. After hybridization to the target cpn60 amplification product, the secondary structure of the probe is disrupted and the fluorescent moieties become separated from one another such that after excitation with light of a suitable wavelength, the emission of the first fluorescent moiety can be detected.

The amount of FRET corresponds to the amount of amplification product, which in turn corresponds to the amount of template nucleic acid present in the sample. Similarly, the amount of template nucleic acid corresponds to the amount of microbe present in the sample. Therefore, the amount of FRET produced when amplifying nucleic acid obtained from a biological sample can be correlated to the amount of microbe present in a sample. Typically, the amount of microbe in a sample can be quantitated by comparing the amount of FRET produced from amplification of nucleic acid in the sample to the amount of FRET produced from amplification of nucleic acid obtained from known amounts of the microbe (e.g., a standard curve). Accurate quantitation requires measuring the amount of FRET while amplification is increasing linearly. In addition, there must be an excess of probe in the reaction. Furthermore, the amount of FRET produced in the known samples used for comparison purposes can be standardized for particular reaction conditions, such that it is not necessary to isolate and amplify samples from every microbe for comparison purposes. Quantitation of microbes used in bioterrorism can be valuable prior to an attack as a method of monitoring (e.g., on a day-to-day basis) a threshold level of a microbe, while quantitation of microbes following an attack can determine the magnitude of response necessary as well as monitor the extent of exposure and the effectiveness of clean-up.

As an alternative to FRET, a cpn60 amplification product can be detected using, for example, a fluorescent DNA binding dye (e.g., SYBRGreenI® or SYBRGold® (Molecular Probes)). Upon interaction with an amplification product, such DNA binding dyes emit a fluorescent signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

Melting curve analysis is an additional step that can be included in a cycling profile. Melting curve analysis is based on the fact that a nucleic acid sequence melts at a characteristic temperature (Tm), which is defined as the temperature at which half of the DNA duplexes have separated into single strands. The melting temperature of a DNA molecule depends primarily upon its nucleotide composition. A DNA molecule rich in G and C nucleotides has a higher Tm than one having an abundance of A and T nucleotides. The temperature at which the FRET signal is lost correlates with the melting temperature of a probe from an amplification product. Similarly, the temperature at which signal is generated correlates with the annealing temperature of a probe with an amplification product. The melting temperature(s) of cpn60 probes from an amplification product can confirm the presence or absence of cpn60-containing microbes in a sample, and can be used to quantitate the amount of a particular cpn60-containing microbe. For example, a universal probe that hybridizes to a variable region within cpn60 will have a Tm that depends upon the sequence to which it hybridizes. Thus, a universal probe may have a Tm of 70° C. when hybridized to a cpn60 amplification product generated from one species of microbe (e.g., B. anthracis), but a Tm of 65° C. when hybridized to a cpn60 amplification product generated from a second species of microbe (e.g., Y. pestis). By observing a temperature-dependent, step-wise decrease in fluorescence of a sample as it is heated, the particular cpn60-containing microbe in the sample can be identified and the relative amounts of the microbe in the sample can be determined.

Within each thermocycler run, control samples can be cycled as well. Positive control samples can amplify a control nucleic acid template (e.g., a nucleic acid other than cpn60) using, for example, control primers and control probes. Positive control samples also can amplify, for example, a plasmid construct containing a cpn60 nucleic acid molecule. Such a plasmid control can be amplified internally (e.g., within the sample) or in a separate sample run side-by-side with the test samples. Each thermocycler run also should include a negative control that, for example, lacks cpn60 template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and species-specificity (if desired) and for FRET to occur.

In one embodiment, methods of the invention include steps to avoid contamination. For example, an enzymatic method utilizing uracil-DNA glycosylase is described in U.S. Pat. Nos. 5,035,996, 5,683,896 and 5,945,313, and can be used to reduce or eliminate contamination between one thermocycler run and the next. In addition, standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

It is understood that the present invention is not limited by the configuration of one or more commercially available instruments.

Fluorescent In Situ Hybridization (FISH)

In situ hybridization methods such as FISH also can be used to detect and identify a microbe in a sample. In general, in situ hybridization methods provided herein include the steps of fixing the contents of a sample, hybridizing a cpn60 probe to DNA contained within the fixed sample, washing to remove non-specific binding, detecting the hybridized probe, and quantitating the amount of hybridized probe.

Typically, microbes are obtained, from a sample using methods described herein. For example, microbes can be harvested by centrifuging a sample and resuspending the pelleted microbes in, for example, phosphate-buffered saline (PBS). After re-centrifuging the microbe suspension to obtain a pellet, the microbes can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the microbes can be prepared by removing a majority of the fixative, leaving the concentrated microbes suspended in only a portion of the solution.

The microbe suspension is applied to slides such that the microbes do not overlap on the slide. Density can be measured by a light or phase contrast microscope. If the well containing the greatest dilution does not have enough cells, the suspension can be concentrated and placed in another well.

Oligonucleotides probes for FISH are chosen for maximal sensitivity and specificity. Using a set of oligonucleotide probes (e.g., two or more cpn60 probes) can provide greater sensitivity and specificity than the use of any one probe. Probes typically are about 50 to about 2×10³ nucleotides in length (e.g., 50, 75, 100, 200, 300, 400, 500, 750, 1000, 1500, or 2000 nucleotides in length). Probes that hybridize to specific DNA can be obtained commercially from, for example, Vysis, Inc. (Downers Grove, Ill.), Molecular Probes, Inc. (Eugene, Oreg.), or from Cytocell (Oxfordshire, UK). Alternatively, oligonucleotide probes for FISH can be made non-commercially from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via PCR. See, for example, Nath and Johnson, Biotechnic Histochem., 1998, 73(1):6-22, Wheeless et al., Cytometry, 1994, 17:319-326, and U.S. Pat. No. 5,491,224.

Oligonucleotide probes for FISH typically are directly labeled with a fluorescent moiety (also referred to as a fluorophore), an organic molecule that fluoresces after absorbing light of lower wavelength/higher energy. The fluorescent moiety allows the probe to be visualized without a secondary detection molecule. After covalently attaching a fluorophore to a nucleotide, the nucleotide can be directly incorporated into a probe using standard techniques such as nick translation, random priming, and PCR labeling. Alternatively, deoxycytidine nucleotides within a probe can be transaminated with a linker. A fluorophore then can be covalently attached to the transaminated deoxycytidine nucleotides. See, U.S. Pat. No. 5,491,224. The amount of fluorophore incorporated into a probe can be known or determined, and this value in turn can be used to determine the amount of nucleic acid to which the probe binds. In conjunction with analysis of samples containing known numbers of microbes (e.g., serial dilutions), the number of microbes in a biological or non-biological sample can be determined.

When more than one oligonucleotide probe is used, fluorescent moieties of different colors can be chosen such that each probe in the set can be distinctly visualized and quantitated. For example, a combination of the following fluorophores may be used: 7-amino-4-methylcoumarin-3-acetic acid (AMCA), Texas Red™ (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc.). Probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine and quantitate hybridization of the probes.

Oligonucleotide probes also can be indirectly labeled with biotin or digoxygenin, or labeled with radioactive isotopes such as ³²P and ³H, although secondary detection molecules or further processing may be required to visualize the probes and quantitate the amount of hybridization. For example, a probe indirectly labeled with biotin can be detected and quantitated using avidin conjugated to a detectable enzymatic marker such as alkaline phosphatase or horseradish peroxidase. Enzymatic markers can be detected and quantitated in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the oligonucleotide probes and the chromosomal DNA contained within the microbes each are denatured. Denaturation typically is performed by incubating in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3 M sodium chloride and 0.03 M sodium citrate). Probes can be denatured by heat (e.g., by heating to about 73° C. for about five minutes).

After removal of denaturing chemicals or conditions, oligonucleotide probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability of forming a hybrid. For example, in situ hybridizations typically are performed in hybridization buffer containing 1-2×SSC, 50% formamide, and blocking DNA to suppress non-specific hybridization. In general, hybridization conditions, as described above, include temperatures of about 25° C. to about 55° C., and incubation times of about 0.5 hours to about 96 hours. More particularly, hybridization can be performed at about 32° C. to about 40° C. for about 2 to about 16 hours.

Non-specific binding of oligonucleotide probes to DNA outside of the target region can be removed by a series of washes. The temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

mRNA-Based Assays

Alternatively, in order to test for the presence or absence of, or measure the level of, a cpn60 mRNA in a sample, e.g., a sample comprising microbes, the microbes can be lysed and total RNA can be purified or semi-purified from lysates by any of a variety of methods known in the art. Methods of detecting or measuring levels of particular mRNA transcripts are also familiar to those in the art. Such assays include, without limitation, hybridization assays using detectably labeled cpn60 nucleic acid (DNA or RNA) probes and quantitative or semi-quantitative RT-PCR methodologies employing appropriate cpn60 oligonucleotide primers. Additional methods for quantitating mRNA in lysates include RNA protection assays and serial analysis of gene expression (SAGE). Alternatively, qualitative, quantitative, or semi-quantitative in situ hybridization assays can be carried out using, for example, samples such as tissue sections or unlysed microbial suspensions, and detectably (e.g., fluorescently, isotopically, or enzymatically)-labeled DNA or RNA probes.

Polypeptide-Based Assays

The invention also features polypeptide-based assays. A cpn60 protein, or cpn60 polypeptide, can be used as a universal target to determine the presence or absence of one or more microbes, and further used as species-specific targets and/or probes for the identification and classification of specific microbes. Such assays can be used on their own or in conjunction with other procedures (e.g., nucleic acid-based assays).

In the assays of the invention, the presence or absence of a cpn60 polypeptide is detected and/or its level is measured. Methods of detecting or measuring the levels of a protein of interest (e.g., a cpn60 protein, or cpn60 polypeptides) in microbes are known in the art. Many such methods employ antibodies (e.g., polyclonal antibodies or mAbs) that bind specifically to the protein.

Antibodies and Antibody-Based Assays

Antibodies having specific binding affinities for a cpn60 protein or a cpn60 polypeptide may be produced through standard methods. As used herein, the terms “antibody” or “antibodies” include intact molecules as well as fragments thereof which are capable of binding to an epitopic determinant of a cpn60 polypeptide. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The terms “antibody” and “antibodies” include polyclonal antibodies, monoclonal antibodies, humanized or chimeric antibodies, single chain Fv antibody fragments, Fab fragments, and F(ab)₂ fragments.

Antibodies may be specific for a particular cpn60 polypeptide, e.g., the cpn60 protein of the B. anthracis microbe. Alternatively, they may be cross-reactive with two or more cpn60 polypeptides, e.g., cross-react or bind to two or more cpn60 proteins. For example, such antibodies may bind to common epitopes present in two or more cpn60 proteins or cpn60 polypeptides. As used herein, such antibodies with specificity for two or more cpn60 polypeptides are termed “universal” antibodies. For example, certain antibodies may bind to common epitopes present in all or most cpn60 polypeptides.

In certain embodiments of the method described herein, it may be sufficient to determine simply whether or not any microbes used in bioterrorism are present, and optionally the relative concentration or amount of such microbes. Such detection may occur through, e.g., the use of one or more “universal” cpn60 antibodies, such as an antibody that binds or demonstrates specificity to two or more cpn60 polypeptides as described previously. Universal antibodies can be used to monitor an environment prior to an attack to determine when an attack has taken place.

In other embodiments, the identification of the particular microbe may be preferred. Identification of the species of microbe used in a bioterrorism attack can determine the size of the response and the methods of containment and clean-up. Accordingly, an antibody specific for a particular microbial species' cpn60 polypeptide may be employed; such antibodies are referred to as having binding affinity for that cpn60 polypeptide. The universal and specific antibodies may be employed simultaneously or in series. For example, a universal antibody may be used as a first screen to determine the presence or absence of a cpn60 polypeptide. Subsequently, a specific antibody, such as one specific for a cpn60 polypeptide from a particular microbe, e.g., B. anthracis, may be employed. In such assays, monoclonal antibodies may be particularly useful (e.g., sensitive) to identify cpn60 polypeptides of a particular species of microbe.

In general, a protein of interest (e.g., a cpn60 protein against which one wishes to prepare antibodies) is produced recombinantly, by chemical synthesis, or by purification of the native protein, and then used to immunize animals. As used herein, an intact cpn60 protein may be employed, or a cpn60 polypeptide may be employed, provided that the cpn60 polypeptide is capable of generating the desired immune response. See, for example, WO 02/65129 for a description of epitopic sequences from Chlamydia trachomatis that are recognized by human antibodies. See also U.S. Pat. No. 6,497,880, which sets forth nucleic acid sequences, amino acid sequences, expression vectors, purified proteins, antibodies, etc. specific to Aspergillus fumigatus and Candida glabrata. Purified Aspergillus fumigatus and Candida glabrata cpn60 proteins, or proteolytically or synthetically generated fragments thereof, can be used to immunize animals to generate antibodies for use in the methods of the present invention. Finally, see WO 02/57784, which discloses substantially purified Chlamydia hsp60 (cpn60) polypeptides. Such polypeptides may also be used to generate antibodies for use in the methods of the present invention.

As discussed previously, one may wish to prepare universal or specific antibodies that have binding affinity to cpn60 proteins or polypeptides. A cpn60 polypeptide from one species of microbe may be used to generate a universal antibody, for example, if that cpn60 polypeptide maintains an epitope that is common to cpn60 proteins from at least one other species of microbe, or, e.g., to all cpn60 proteins that one wishes to detect (e.g., the cpn60 proteins from species of microbes used in bioterrorism). Alternatively, a cpn60 protein or cpn60 polypeptide may be used to generate antibodies specific for the cpn60 protein or polypeptide from a particular microbe, e.g., only in B. anthracis.

Various host animals including, for example, rabbits, chickens, mice, guinea pigs, and rats, can be immunized by injection of the cpn60 protein(s) of interest. Adjuvants can be used to increase the immunological response depending on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin (KLH), and dinitrophenol. Polyclonal antibodies are heterogenous populations of antibody molecules that are specific for a particular antigen, which are contained in the sera of the immunized animals. Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler et al., 1975, Nature, 256:495, the human B-cell hybridoma technique (Kosbor et al., 1983, Immunology Today, 4:72; Cole et al., 1983, Proc. Natl. Acad. Sci. USA, 80:2026), and the EBV-hybridoma technique (Cole et al., “Monoclonal Antibodies and Cancer Therapy”, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a murine monoclonal antibody and a constant region derived from a human immunoglobulin. Chimeric antibodies can be produced through standard techniques.

Antibody fragments that have specific binding affinity for a cpn60 polypeptide can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

Once produced, antibodies or fragments thereof are tested for recognition of a cpn60 protein or cpn60 polypeptide by standard immunoassay methods including, for example, ELISA techniques, countercurrent immuno-electrophoresis (CIEP), radioimmunassays (RIA), radioimmunoprecipitations, dot blots, inhibition or competition assays, sandwich assays, immunostick (dipstick) assays, immunochromatographic assays, immunofiltration assays, latex beat agglutination assays, immunofluorescent assays, biosensor assays. See, Short Protocols in Molecular Biology, Chapter 11, Green Publishing Associates and John Wiley & Sons, Edited by Ausubel, F. M et al., 1992; Antibodies: A Laboratory Manual, Harlow and Lane (eds.), Cold Spring Harbor Laboratory Press, 1988; and U.S. Pat. Nos. 4,376,110; 4,486,530; and 6,497,880. Antibodies or fragments can also be tested for their ability to react universally, e.g., with cpn60 proteins or cpn60 polypeptides from more than one species of microbe, (e.g., the cpn60 proteins from B. anthracis and C. botulinum), or specifically with a particular species' cpn60 protein (e.g., the cpn60 protein of B. anthracis).

In antibody assays, the antibody itself or a secondary antibody that binds to the primary antibody can be detectably labeled. Alternatively, the antibody can be conjugated with biotin, and detectably-labeled avidin can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. The methods described below for detecting a cpn60 polypeptide in a liquid sample can also be used to detect a cpn60 polypeptide in lysates.

Methods of detecting a cpn60 polypeptide in a liquid sample generally involve contacting a sample of interest with an antibody that binds to a cpn60 polypeptide and determining whether or not the antibody bound to a component of the sample. In such assays the antibody need not be detectably labeled and can be used without a second antibody that binds to a cpn60 polypeptide. For example, an antibody with binding affinity for a cpn60 polypeptide may be bound to an appropriate solid substrate and then exposed to the sample. Binding of a cpn60 polypeptide to the antibody on the solid substrate may be detected by exploiting the phenomenon of surface plasmon resonance, wherein binding results in a change in the intensity of surface plasmon resonance that can be detected qualitatively or quantitatively by an appropriate instrument, (e.g., a Biacore apparatus (Biacore International AB, Rapsgatan, Sweden).

Moreover, assays for detection of a cpn60 polypeptide in a liquid sample can involve the use, for example, of: (a) a detectably-labeled single antibody specific for a cpn60 polypeptide; (b) an unlabeled antibody that is specific for a cpn60 polypeptide and a detectably-labeled secondary antibody; or (c) a biotinylated antibody specific for a cpn60 polypeptide and detectably-labeled avidin. In addition, combinations of these approaches (including “multi-layer” assays) familiar to those in the art can be used to enhance the sensitivity of such assays. In these assays, the sample or an aliquot of the sample suspected of containing a microbe used in bioterrorism can be immobilized on a solid substrate, such as a nylon or nitrocellulose membrane, by, for example, “spotting” an aliquot of the liquid sample or by blotting of a gel on which the sample or an aliquot of the sample has been subjected to electrophoretic separation. The presence or amount of cpn60 polypeptide on the solid substrate is then assayed using any of the above-described forms of a cpn60 polypeptide specific antibody and, where required, appropriate detectably-labeled secondary antibodies or avidin.

The invention also features “sandwich” assays. In sandwich assays, instead of immobilizing samples on solid substrates by the methods described above, any cpn60 polypeptide that may be present in a sample can be immobilized on the solid substrate by conjugating a second (“capture”) antibody (polyclonal or mAb) having binding affinity for a cpn60 polypeptide to the solid substrate prior to exposing the solid substrate to the sample by any of a variety of methods known in the art. Exposing a sample to the solid substrate containing the second antibody specific for cpn60 polypeptide, any cpn60 polypeptide in the sample (or sample aliquot) will bind to the second antibody on the solid substrate. The presence or amount of cpn60 polypeptide bound to the conjugated second antibody is then assayed using a “detection” antibody specific for a cpn60 polypeptide by methods essentially the same as those described above

It is understood that in these sandwich assays, the capture antibody should not bind to the same epitope (or range of epitopes in the case of a polyclonal antibody) as the detection antibody. Thus, if a mAb is used as a capture antibody, the detection antibody can be either: (a) another mAb that binds to an epitope that is either completely physically separated from or only partially overlaps the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope that is either physically separated from or partially overlaps with any of the epitopes to which the capture polyclonal antibody binds; or (b) a polyclonal antibody that binds to epitopes other than or in addition to that to which the capture polyclonal antibody binds. Assays that involve the use of capture and detection antibodies include sandwich ELISAs, sandwich Western blotting assays, and sandwich immunomagnetic detection assays.

Suitable solid substrates to which the capture antibody can be bound include, without limitation, microtiter plates, membranes such as nylon or nitrocellulose membranes, and polymeric (e.g., without limitation, agarose, cellulose, or polyacrylamide) beads or particles. It is noted that antibodies bound to such beads or particles can also be used for immunoaffinity purification of cpn60 polypeptides. Dipstick/immunostick formats can employ a solid phase, e.g., polystyrene, paddle or dispstick.

Methods of detecting or for quantitating a detectable label depend on the nature of the label and are known in the art. Appropriate labels include, without limitation, radionuclides (e.g., ¹²⁵I, ¹³¹I, ³⁵S, ³H, ³²P, ³³P, or ¹⁴C), fluorescent moieties (e.g., fluorescein, rhodamine, or phycoerythrin), luminescent moieties (e.g., Qdot™ nanoparticles supplied by the Quantum Dot Corporation, Palo Alto, Calif.), or enzymes (e.g., alkaline phosphatase or horseradish peroxidase). The products of reactions catalyzed by appropriate enzymes can be, without limitation, fluorescent, luminescent, or radioactive, or they may absorb visible or ultraviolet light. Examples of detectors include, without limitation, x-ray film, radioactivity counters, scintillation counters, spectrophotometers, colorimeters, fluorometers, luminometers, and densitometers.

The methods of the present invention may employ a control sample. In assays to detect the presence or absence of a microbe used in bioterrorism, the concentration of a cpn60 polypeptide in, for example, a food sample suspected of being contaminated, or at risk of being contaminated, with, for example, C. botulinum microbes that produce botulinum toxin may be compared to a control sample, e.g., a food sample known not to be infected. The control sample may be taken from the same or a closely related environment, e.g., in a different location known to be uncontaminated, or can be a control sample taken from an environment completely removed from the putative bioterrorism attack (e.g., an equivalent sample from a different building). Alternatively, the control sample may be taken from the same location but at an earlier or later time-point when the location was known to be uncontaminated. A significantly higher concentration of cpn60 polypeptide in the suspect sample relative to the control sample would indicate the presence of one or more microbes used in bioterrorism.

It is understood that, while the above descriptions of the diagnostic assays may refer to assays on food samples or bodily fluid samples, the assays can also be carried out on any of the other fluid or solubilized samples listed herein, such as water samples, air samples, or buffer samples (e.g., buffer used to extract a sample from a fomite).

Other Polypeptide-Based Detection Assays

The present invention also contemplates the use of other analytical techniques for detecting the presence, absence, or amount of cpn60 polypeptides. Recent analytical instrumentation and methodology advances that have arisen in the context of proteomics research are applicable in the methods of the present invention. See, generally, Jungblut, 2001, Microbes & Infection, 3:831-840; MacBeath and Schreiber, 2000, Science, 289:1760-1763; Madoz-Gdrpide et al., 2001, Proteomics, 1: 1279-1287; Patterson, 2000, Physiological Genomics, 2:59-65; and Schevchenko et al., 2000, Analytical Chemistry, 72:2132-2141.

Mass-spectrophotometric techniques have been increasingly used to detect and identify proteins and protein fragments at low levels, e.g., fmol or pmol. Mass spectrometry has become a major analytical tool for protein and proteomics research because of advancements in the instrumentation used for biomolecular ionization, electrospray ionization (ESI), and matrix-assisted laser desorption-ionization (MALDI). MALDI is usually combined with a time-of-flight (TOF) mass analyzer. Typically, 0.5 μl of sample that contains 1-10 pmol of protein or peptide is mixed with an equal volume of a saturated matrix solution and allowed to dry, resulting in the co-crystallization of the analyte with the matrix. Matrix compounds that are used include sinapic acid and α-hydroxycinnamic acid. The co-crystallized material on the target plate is irradiated with a nitrogen laser pulse, e.g., at a wavelength of 337 nm, to volatilize and ionize the protein or peptide molecules. A strong acceleration field is switched on, and the ionized molecules move down the flight tube to a detector. The amount of time required to reach the detector is related to the mass-to-charge ratio. Proteolytic mass mapping and tandem mass spectrometry, when combined with searches of protein and protein fragment databases, can also be employed to detect and identify cpn60 polypeptides. See, for example, Downard, 2000, J. Mass. Spectrom. 35:493-503.

Biomolecular interaction analysis mass spectrometry (BIA-MS) is another suitable technique for detecting interactions between cpn60 polypeptides and cpn60 antibodies. This technology detects molecules bound to a ligand that is covalently attached to a solid surface. As the density of biomaterial on the surface increases, changes occur in the refractive index at the solution or surface interface. This change in the refractive index is detected because the angle or wavelength at which the incident light is absorbed at the surface changes upon binding. The difference in the angle or wavelength is proportional to the amount of material bound on the surface, giving rise to a signal that is termed surface plasmon resonance (SPR), as discussed previously. See, for example, Nelson et al., 1999, Analytical Chemistry, 71:2858-2865; see also Nedelkov and Nelson, Biosensors and Bioelectronics 16:1071-1078.

The SPR biosensing technology has been combined with MALDI-TOF mass spectrometry for the desorption and identification of biomolecules. In a chip-based approach to BIA-MS, a ligand, e.g., a cpn60 antibody, is covalently immobilized on the surface of a chip. A tryptic digestion of solubilized proteins from a sample is routed over the chip, and the relevant peptides, e.g., cpn60 polypeptides, are bound by the ligand. After a washing step, the eluted peptides are analyzed by MALDI-TOF mass spectrometry. The system may be a fully automated process and is applicable to detecting and characterizing proteins present in complex biological fluids and cell extracts at low- to sub-femtomol levels.

Mass spectrometers useful for such applications are available from Applied Biosystems (Foster City, Calif.); Bruker Daltronics (Billerica, Mass.) and Amersham Pharmacia (Sunnyvale, Calif.).

Other suitable techniques for use in the present invention include “Multidimensional Protein Identification Technologies.” Cells are fractionally solubilized and digested, e.g., sequentially with endoproteinase Lys-C and immobilized trypsin. The samples are then subjected to multidimensional protein identification technology (MUDPIT), which involves a sequential separation of the peptide fragments by on-line biphasic microcapillary chromatography (e.g., strong ion exchange, then C-18 separation), followed by tandem mass spectrometry (MS-MS). See, for example, Washburn et al., 2001, Nature Biotechnology, 19:242-247.

Articles of Manufacture

The invention also provides articles of manufacture. Articles of manufacture can include at least one cpn60 oligonucleotide primer, as well as instructions for using the cpn60 oligonucleotide(s) to identify and quantitate the amount of one or more microbes in a sample. Typically, the instructions also will relate to use of the reagents in a bioterrorism context such as a threat or actual emergency.

In one embodiment, the cpn60 oligonucleotide(s) are attached to a microarray (e.g., a GeneChip®, Affymetrix, Santa Clara, Calif.). In another embodiment, an article of manufacture can include one or more cpn60 oligonucleotide primers and one or more cpn60 oligonucleotide probes. Such cpn60 primers and probes can be used, for example, in real-time amplification reactions to amplify and simultaneously detect cpn60 amplification products.

Suitable oligonucleotide primers include those that are complementary to highly conserved regions of cpn60 and that flank a variable region. Such universal cpn60 primers can be used to specifically amplify these variable regions, thereby providing a sequence with which to identify microbes used in bioterrorism. Examples of universal cpn60 oligonucleotide primers include the following:

| | |
|---|---|
| 5′-GAIIIIGCIGGIGA(T/C)GGIACIACIAC-3′; | (SEQ ID NO:1) and |
| 5′-(T/C)(T/G)I(T/C)(T/G)ITCICC(A/G)AAICCIGGIGC(T/C)TT-3′. | (SEQ ID NO:2) |

Suitable oligonucleotide primers also include those that are complementary to species-specific cpn60 sequences, and thus result in an amplification product only if a particular species of microbe is present in the sample. In another embodiment, an article of manufacture of the invention can include a full complement of species-specific cpn60 nucleic acids to detect any microbe that is potentially or traditionally used in bioterrorism. For example, cpn60 nucleic acids that are specific to each of B. anthracis, C. botulinum, Y. pestis, and F. tularensis can be included in an article of manufacture of the invention. Such species-specific cpn60 nucleic acids can be used in separate but simultaneous reactions to not only detect the presence or absence of a microbe used in bioterrorism, but also to identify which microbe is present in the sample being tested.

Similar to cpn60 oligonucleotide primers, cpn60 oligonucleotide probes generally are complementary to cpn60 sequences. cpn60 oligonucleotide probes can be designed to hybridize universally to cpn60 sequences from more than one species of microbe, or cpn60 oligonucleotide probes can be designed for species-specific hybridization to, for example, the variable region of cpn60 nucleic acids.

An article of manufacture of the invention can further include additional components for carrying out amplification reactions and/or reactions, for example, on a microarray. Articles of manufacture for use with PCR reactions can include nucleotide triphosphates, an appropriate buffer, and a polymerase. An article of manufacture of the invention also can include appropriate reagents for detecting amplification products. For example, an article of manufacture can include one or more restriction enzymes for distinguishing amplification products from different species of microbe, or can include fluorophore-labeled oligonucleotide probes for real-time detection of amplification products.

It will be appreciated by those of ordinary skill in the art that different articles of manufacture can be provided to evaluate different microbes used in bioterrorism. For example, an article of manufacture can be designed to detect, for example, B. anthracis. Alternatively, a more generalized article of manufacture can be used to evaluate a number of, or all of, the microbes used in bioterrorism.

Articles of manufacture also or alternatively can include at least one cpn60 antibody, as well as instructions for using the same to detect the presence of a species of microbe, and optionally to identify the species of microbe in a sample. In one embodiment, one or more cpn60 antibodies are attached to a microarray (e.g., a 96-microwell plate). For example, a microarray format may include a variety of universal and species-specific cpn60 capture antibodies; the universal and species-specific antibodies can each be located at a different well location. The article of manufacture may also include the appropriate detection antibodies, if necessary, and appropriate reagents for detection of binding of a cpn60 polypeptide to one or more capture antibodies (e.g., enzymes, substrates, buffers, and controls).

In another embodiment, an article of manufacture can include one or more cpn60 antibodies attached to a dipstick. Such dipsticks can be used, for example, to detect cpn60 polypeptides in a sample (e.g., a liquid sample).

The invention will be further described in the following examples, which do not limit the scope of the invention described in the claims.

### EXAMPLES

### Example 1

### Quantitation of Microbes Using Universal Primers and a Universal Probe

A sample is obtained from a swab of a food preparation surface in a restaurant and genomic DNA is extracted using standard methods (Diagnostic Molecular Microbiology: Principles and Applications (supra)). Real-time PCR is conducted using universal cpn60 primers having the nucleotide sequences set forth in SEQ ID NO:1 and SEQ ID NO:2, and a universal cpn60 probe having the sequence 5′-GAC AAA GTC GGT AAA GAA GGC GTT ATC A-3′ (SEQ ID NO:3), labeled at the 5′ end with fluorescein (fluorophore; Molecular Probes, Inc.) and at the 3′ end with DABCYL (quencher; (4-(4′-dimethylaminophenylazo)benzoic acid) succinimidyl ester; Molecular Probes, Inc.). This probe binds to a variable region of the cpn60 gene from numerous microbial species including Salmonella spp. and E. coli; thus the Tm of the probe from an amplification product varies depending upon the nucleotide sequence within the amplification product to which the probe hybridizes.

The PCR reaction contains 3 μL extracted DNA, 1 μM each universal cpn60 primer, 340 nM universal cpn60 probe, 2.5 units Amplitaq Gold DNA polymerase (Perkin Elmer), 0.25 mM each deoxyribonucleotide, 3.5 mM MgCl₂, 50 mM KCl, and 10 mM Tris-HCl (pH 8.0) in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles. After the cycling steps are complete, the melting temperature of the universal probe from the amplification products is analyzed. As the temperature is increased, the universal probe is released from the amplification product from each species' cpn60 sequence at a specific temperature, corresponding to the Tm of the universal probe and the cpn60 sequence of the particular species. The step-wise loss of fluorescence at particular temperatures is used to identify the particular species or microbe present, and the loss in fluorescence of each step compared to the total amount of fluorescence correlates with the relative amount of each particular microbe.

### Example 2

### Quantitation of Microbes Using Universal Primers and Species-Specific Probes

A sample is obtained from food served at a National Football League (NFL) game and nucleic acids are extracted using standard methods (Diagnostic Molecular Microbiology: Principles and Applications (supra)). Real-time PCR is conducted using universal cpn60 primers having the nucleotide sequences set forth in SEQ ID NO:1 and SEQ ID NO:2, and species-specific cpn60 oligonucleotide probes that hybridize to cpn60 nucleotide sequences from, and distinguish between, C. botulinum antigenic types A-G.

The sequences of the probes are identified by aligning cpn60 sequences from the seven antigenic types of C. botulinum and identifying a sequence that is specific to each antigenic type (i.e., a sequence not found in the other antigenic types). Each of the species-specific probes is labeled with a different fluorescent moiety to allow differential detection of the various species.

The PCR reaction contains 3 μL extracted DNA, 1 μM each universal cpn60 primer, 340 nM universal cpn60 probe, 2.5 units Amplitaq Gold DNA polymerase (Perkin Elmer), 0.25 mM each deoxyribonucleotide, 3.5 mM MgCl₂, 50 mM KCl, and 10 mM Tris-HCl (pH 8.0) in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles, at wavelengths corresponding to the particular moieties on the probes. The amount of fluorescence detected at each of the monitored wavelengths correlates with the amount of each cpn60 amplification product. The amount of each antigenic type-specific amplification product is then correlated with the amount of each antigenic type of C. botulinum by comparison to the amount of amplification product generated from samples containing nucleic acid isolated from known amounts of each antigenic type of C. botulinum.

### Example 3

### Dipstick ELISA assay for B. anthracis

A polystyrene dipstick containing two horizontal bands is constructed: one band consists of highly specific, monoclonal capture antibodies against cpn60 proteins from B. anthracis, while the other band is an internal control consisting of horseradish peroxidase. A sample is collected from a filter in the ventilation system of a high-rise office building. The material collected from the filter is resuspended in PBS buffer and serial dilutions are made (1:2, 1:5, 1:10, etc.) directly into a detection reagent. A wetted dipstick is incubated in these dilutions for 5 minutes, and an indicator is then added to detect binding of cpn60 proteins to the capture antibodies. The detection reagent includes a suitable buffer and secondary IgG detection antibodies labeled with horseradish peroxidase. The indicator can be a chromogenic horseradish peroxidase substrate, such as 2,2′-AZINO-bis 3-ethylbenziazoline-6-sulfonic acid, or ABTS. ABTS is considered a safe, sensitive substrate for horseradish peroxidase that produces a blue-green color upon enzymatic activity that can be quantitated at 405-410 nm. At the end of the incubation and indicator steps, the dipstick is rinsed with water (e.g., deionized water) and examined for staining of the antibody band by visual inspection. Staining of the antibody band reveals the presence of B. anthracis in the sample. The internal control band provides a check on the integrity of the detection reagent.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting the presence, absence, or amount of one or more microbes used in bioterrorism, said method comprising:
a) providing a sample obtained from an environment susceptible to bioterrorism attack or an environment within which a bioterrorism attack has taken place; and
b) detecting the presence, absence, or amount of a cpn60 marker in said sample, wherein said cpn60 marker is specific for one or more of said microbes used in bioterrorism,
wherein the presence of said cpn60 marker in said sample is indicative of the presence, in said environment, of one or more of said microbes used in bioterrorism.

2. The method of claim 1, wherein said detecting step is capable of identifying said microbe(s) in said sample.

3. The method of claim 1, wherein said detecting step is capable of quantitating said microbe(s) in said sample.

4. The method according to claim 1, wherein said sample is provided at two or more points.

5. The method according to claim 4, wherein said two or more points are time points.

6. The method according to claim 4, wherein said two or more points are location points.

7. The method according to claim 4, further comprising comparing said presence, absence, or amount of said microbe at said two or more points.

8. The method according to claim 1, further comprising acquiring a control sample from one or both of said environments.

9. The method according to claim 8, further comprising comparing the presence, absence, or amount of microbes used in bioterrorism in said control sample with said presence, absence, or amount of said microbes used in bioterrorism in said sample.

10. The method according to claim 1, wherein said cpn60 marker is a cpn60 nucleic acid or a cpn60 polypeptide.

11. The method according to claim 10, wherein said cpn60 nucleic acid is a nucleic acid coding sequence of a cpn60 protein.

12. The method according to claim 10, wherein said cpn60 nucleic acid is an amplified sequence of a cpn60 coding sequence from said microbes used in bioterrorism.

13. The method according to claim 1, wherein said detecting step is a nucleic acid-based assay.

14. The method according to claim 13, wherein said nucleic acid-based assay is selected from the group consisting of PCR and FISH assays.

15. The method according to claim 1, wherein said detecting step is a polypeptide-based assay.

16. The method according to claim 15, wherein said polypeptide-based assay is an immunodiagnostic assay.

17. The method according to claim 16, wherein said immunodiagnostic assay is ELISA.

18. The method according to claim 15, wherein said polypeptide-based assay comprises a mass spectrometric technique.

19. The method according to claim 15, wherein said polypeptide-based assay comprises a surface plasmon resonance technique.

20. The method according to claim 1, wherein said microbe used in bioterrorism is selected from the group consisting of B. anthracis, C botulinum, Y. pestis, and F. tularensis.

21. The method according to claim 1, wherein said environment susceptible to bioterrorism attack is selected from the group consisting of a food-service facility, a water facility, a transportation facility, an entertainment facility, a shopping mall, and an office building.

22. The method according to claim 21, wherein said food-service facility is selected from the group consisting of a restaurant, a cafeteria, a snack bar, and a convenience store.

23. The method according to claim 21, wherein said water facility is selected from the group consisting of potable water facilities, desalinization facilities, dams, recycled water facilities, water storage tanks, and potable water reservoirs.

24. The method according to claim 21, wherein said transportation facility is selected from the group consisting of an airport, a bus terminal, a train terminal, a port, a Custom's checkpoint, and an immigration checkpoint.

25. The method according to claim 21, wherein said entertainment facility is selected from the group consisting of a club, a theater, a stadium, and an arena.

26. The method according to claim 1, wherein said sample is a biological sample.

27. The method according to claim 26, wherein said biological sample is water.

28. The method according to claim 26, wherein said biological sample is air.

29. The method according to claim 26, wherein said biological sample is food.

30. The method according to claim 26, wherein said biological sample is a tissue sample from an animal or human.

31. The method according to claim 26, wherein said biological sample is a fluid sample from an animal or human.

32. The method according to claim 1, wherein said sample is a non-biological sample.

33. The method according to claim 32, wherein said non-biological sample is collected from a fomite.

34. An article of manufacture, comprising:
at least one cpn60 antibody, wherein said cpn60 antibody has specific binding affinity for a cpn60 polypeptide from a microbe used in bioterrorism, wherein said cpn60 antibody is attached to a solid support; and
an indicator molecule.

35. The article of manufacture of claim 34, further comprising instructions for using said cpn60 antibody to detect a cpn60-containing microbe used in bioterrorism.

36. The article of manufacture of claim 34, wherein said solid support is a dipstick.

37. An article of manufacture, comprising:
at least one cpn60 oligonucleotide, wherein said cpn60 oligonucleotide is complementary to cpn60 nucleic acids from at least one microbe used in bioterrorism; and
instructions therein for using said cpn60 oligonucleotide(s) to determine, in a sample, the presence, absence, or amount of a microbe used in bioterrorism.

38. The article of manufacture of claim 37, wherein said cpn60 oligonucleotide(s) are attached to a microarray.
